(19) United States
(12) Patent Application Publication
(10) Pub. No.: US 2014/0074048 A1
(43) Pub. Date: Mar. 13, 2014

(54) INTRAVENOUS FLOW CONTROL DEVICE

(71) Applicant: Hsien-Tsung WANG, Taipei (TW)

(72) Inventor: Hsien-Tsung WANG, Taipei (TW)

(21) Appl. No.: 13/610,898

(22) Filed: Sep. 12, 2012

(51) Int. Cl. A61M 39/22

(52) U.S. Cl. 604/256

(57) ABSTRACT

An intravenous flow control device includes a rigid casing (2) defining a flow passage having a fluid entrance (211) and a fluid exit (222); and a flow regulator (1) disposed in the casing (2) and including a floating member (11) having a hollow cylinder (111), an opening (112) on a bottom of the hollow cylinder (111), and a lower stepped-diameter chamber (113) in the hollow cylinder (111) and communicating with the opening (112); and a valve member (12) including a hollow member (121), a flat member (122), and a space (123) open to top. The valve member (12) is retained in the chamber (113) through the opening (112) and together they form a sealed hollow structure. The flow regulator (1) floats on fluid in the casing (2) due to the sealed hollow structure.

### BACKGROUND OF THE INVENTION

1. Field of the Invention

The invention relates to intravenous (IV) infusion and more particularly to an intravenous flow control device.

2. Description of Related Art

Conventionally, an IV infusion set comprises a flow control device and a manual flow control device together for adjusting IV fluids flowing to a patient or stopping the infusion at the end of IV infusion. Also, they can prevent air from entering the blood stream (i.e., air embolism).

The conventional flow control device comprises a float buoyed in a casing containing IV fluid, and a suction cup member under the float. However, the float may not function normally and/or the suction cup member may not be capable of blocking an outlet if the flow control device is inclined.

Thus, the need for improvement still exists.

### SUMMARY OF THE INVENTION

It is therefore one object of the invention to provide an intravenous flow control device comprising a flow regulator disposed in a casing and comprising a floating member including a hollow cylinder, an opening on a bottom of the hollow cylinder, and a lower stepped-diameter chamber in the hollow cylinder and communicating with the opening; and a valve member including a hollow member, a flat member, and a space open to top; wherein the valve member is retained in the chamber through the opening and together they form a sealed hollow structure; and wherein the flow regulator floats on fluid in the casing due to the sealed hollow structure.

The above and other objects, features and advantages of the invention will become apparent from the following detailed description taken with the accompanying drawings.

### BRIEF DESCRIPTION OF THE DRAWINGS

FIG. 1 is a perspective view of an intravenous flow control device according to a first preferred embodiment of the invention;

FIG. 2 is an exploded view of FIG. 1;

FIG. 3A is an enlarged view of a first configuration of a flow regulator of FIG. 2;

FIG. 3B is a longitudinal sectional view of the assembled flow regulator of FIG. 3A;

FIG. 4 is a longitudinal sectional view of FIG. 1 showing fluid dropped into the casing during an IV infusion;

FIG. 5 is a view similar to FIG. 4 showing the IV flow completely blocked by the flow regulator at the end of IV infusion;

FIG. 6A is an enlarged view of a second configuration of a flow regulator of FIG. 2;

FIG. 6B is a longitudinal sectional view of the assembled flow regulator of FIG. 6A;

FIG. 7 is a longitudinal sectional view of FIG. 1 showing fluid dropped into the casing during an IV infusion in which the flow regulator is the second configuration;

FIG. 8 is a view similar to FIG. 7 showing the IV flow completely blocked by the second configuration of the flow regulator at the end of IV infusion;

FIG. 9 is a perspective view of an IV infusion set incorporating the intravenous flow control device according to the first preferred embodiment of the invention;

FIG. 10A is an enlarged view of a first configuration of a flow regulator of an intravenous flow control device according to a second preferred embodiment of the invention;

FIG. 10B is a longitudinal sectional view of the assembled flow regulator of FIG. 10A;

FIG. 11A is an enlarged view of a second configuration of a flow regulator of the intravenous flow control device according to the second preferred embodiment of the invention;

FIG. 11B is a longitudinal sectional view of the assembled flow regulator of FIG. 11A;

FIG. 12 is a longitudinal sectional view of FIG. 1 showing fluid dropped into the casing during an IV infusion in which the flow regulator is the first configuration of the intravenous flow control device according to the second preferred embodiment of the invention;

FIG. 13 is a longitudinal sectional view of FIG. 1 showing fluid dropped into the casing during an IV infusion in which the flow regulator is the second configuration of the intravenous flow control device according to the second preferred embodiment of the invention;

FIG. 14A is an enlarged view of a flow regulator of an intravenous flow control device according to a third preferred embodiment of the invention;

FIG. 14B is a longitudinal sectional view of the assembled flow regulator of FIG. 14A;

FIG. 15 is a longitudinal sectional view of FIG. 1 showing fluid dropped into the casing during an IV infusion in which the flow regulator is the one shown in FIGS. 14A and 14B; and

FIG. 16 is a view similar to FIG. 15 showing the IV flow completely blocked by the flow regulator at the end of IV infusion.

### DETAILED DESCRIPTION OF THE INVENTION

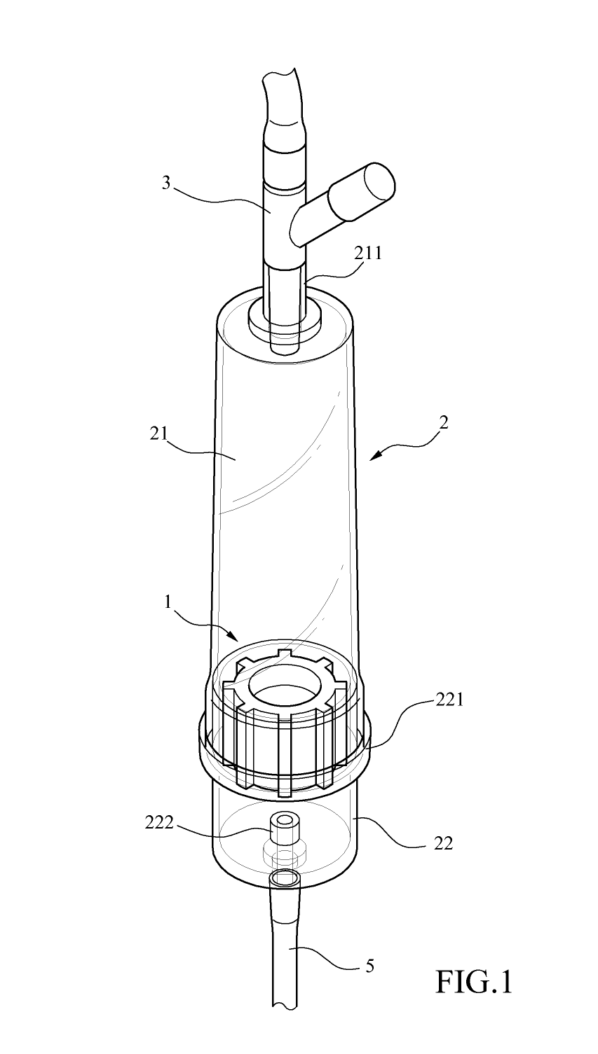

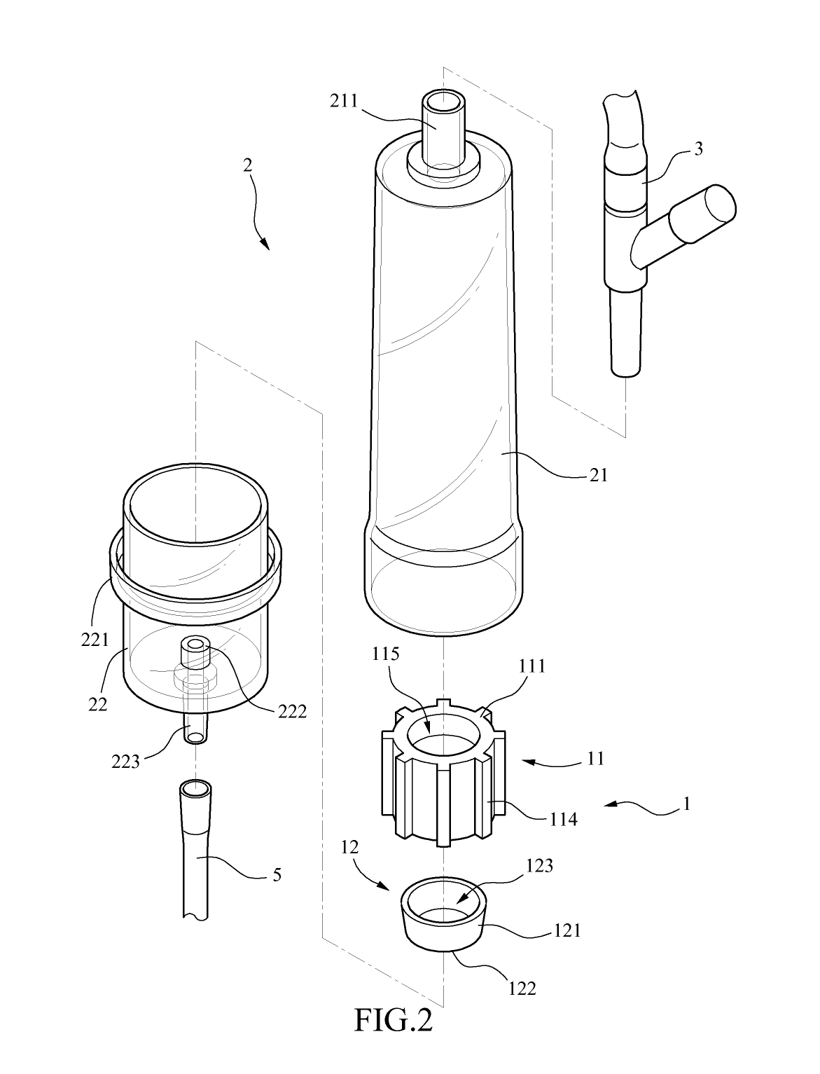

Referring to FIGS. 1 to 9, an IV infusion set in accordance with a first preferred embodiment of the invention is shown. The IV fusion set comprises, from upstream to downstream, a drip chamber 4 filled with fluid, a first plastic IV tubing 3, an flow control device, a second plastic IV tubing 5, a manual flow control device 7, and an IV needle 6. The flow control device as the subject of the invention and its components will be discussed in detail below.

A flow regulator 1 comprising a floating member 11 and a valve member 12 disposed in a rigid, cylindrical, transparent casing 2. The casing 2 comprises an upper member 21 and a lower member 22 fastened together. The upper member 21 comprises a projecting inlet 211 on top. The lower member 22 comprises an intermediate annular grooved flange 221 on an outer surface, together with a portion of the lower member 22 above the flange 221, for fastening a lower portion of the upper member 21 put thereon. The lower member 22 further comprises an inner tubing connector 222 on a bottom and an outer tubing connector 223 in fluid communication with the inner tubing connector 222 and downward projecting out of the bottom. The outer tubing connector 223 is connected to one end of the second plastic IV tubing 5 for allowing fluid communication therewith.

The floating member 11 is a hollow cylindrical member and comprises a hollow cylinder 111, a bottom opening 112 on a bottom of the cylinder 111, a stepped-diameter chamber 113 in a lower portion of the cylinder 111 and communicating with the bottom opening 112, a plurality of equally spaced longitudinal ridges 114 of rectangular cross-section formed on an outer surface of the cylinder 111, and a recess 115 on a top of the cylinder 111. The recess 115 is separated from the chamber 113 by a member. Depth of the recess 115 is about one-fifth of the height of the cylinder 111. The recess 115 is capable of adjusting buoyancy.

Outer diameter of the floating member 11 is slightly less than an inner diameter of each of the upper member 21 and the lower member 22. Thus, the floating member 11 can freely float upward or downward in both the upper member 21 and the lower member 22 (i.e., the casing 2). Thus, the ridges 114 may frictionally contact an inner surface of the casing 2 when the casing is inclined. This has the advantage of keeping the flow regulator 1 from upside down and preventing the flow regulator 1 from being malfunctioned.

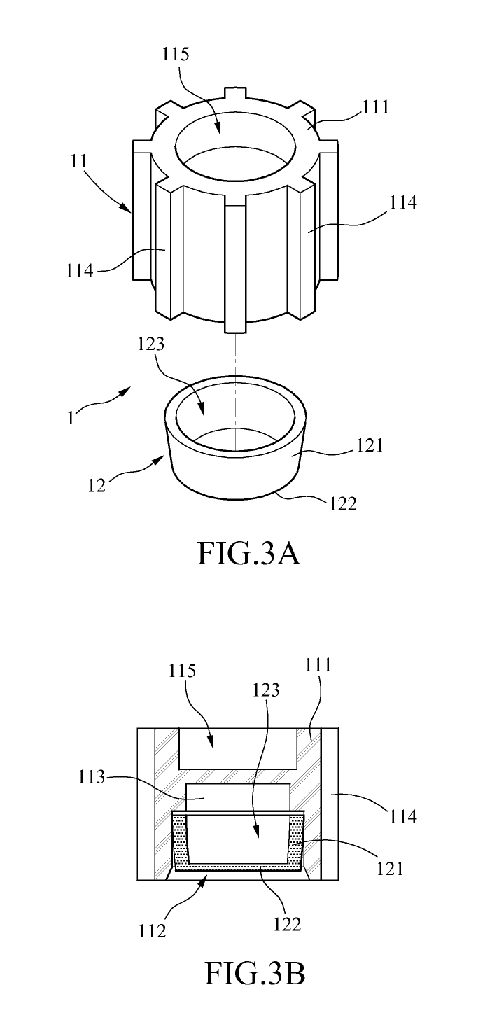

As shown in FIGS. 3A and 3B specifically, in a first configuration the valve member 12 is bowl-shaped and comprises a downward tapered hollow member 121, a flat member 122, and a space 123 open to top. The valve member 12 can be disposed in the chamber 113 through the bottom opening 112.

The valve member 12 can be oriented with top being open as that shown in FIG. 3A prior to disposing in the chamber 113 through the opening 112. Thus, the space 123 in the hollow member 121 can communicate with the chamber 113 of the floating member 11. Therefore the floating member 11 and the valve member 12 having the flat member 122 as a blocking member form a sealed hollow structure.

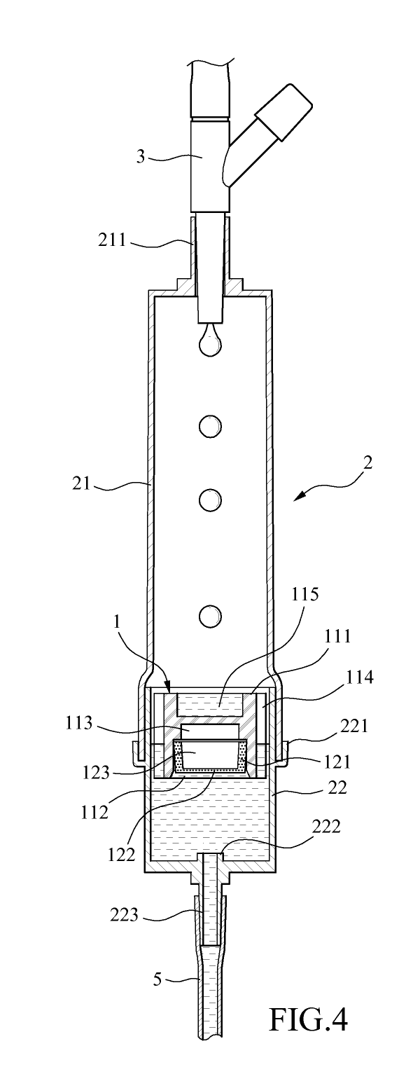

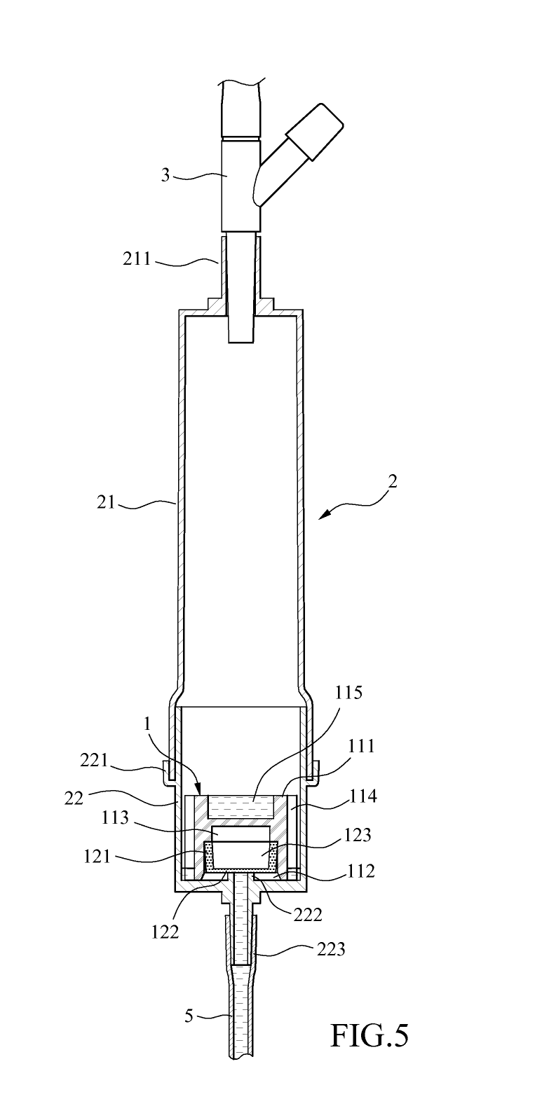

As shown in FIGS. 4 and 5 specifically, fluid can be fed from the drip chamber 4, the first plastic IV tubing 3, and the inlet 211 into the casing 2 to accumulate therein. As such, the floating member 11 and the valve member 12 as a unit float in the casing 2. In FIG. 4, it is shown that the recess 115 is filled with fluid and a portion of space of the casing 2 below the flow regulator 1 is filled with fluid. Level of the fluid in the casing 2 may drop due to fluid flow out of the casing 2 via the inner tubing connector 222, the outer tubing connector 223, and the second plastic IV tubing 5. In a position of FIG. 5, the inner tubing connector 222 is completely blocked by the flat member 122 of the valve member 12. As a result, the fluid flow is stopped.

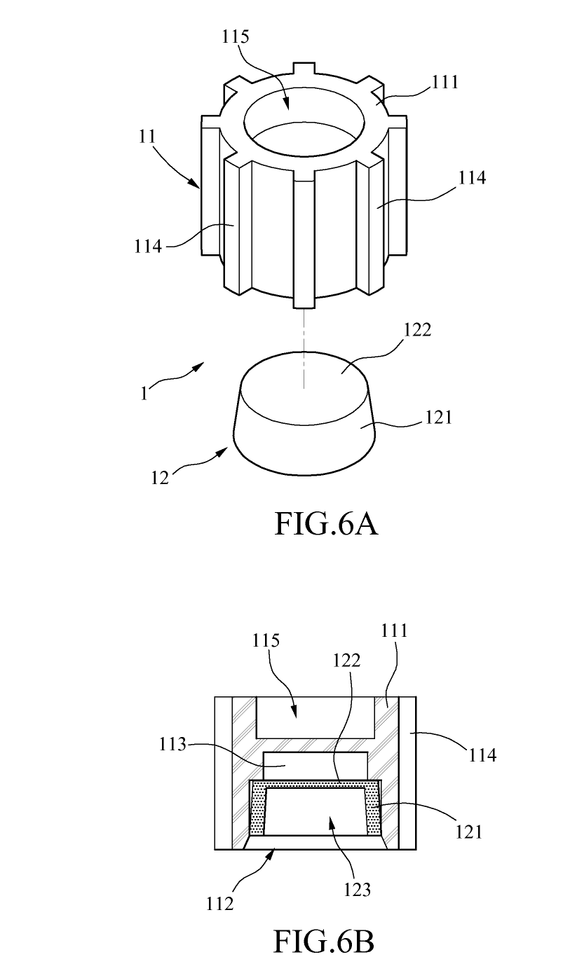

As shown in FIGS. 6A and 6B specifically, in a second configuration the valve member 12 is inverted bowl-shaped and comprises an upward tapered hollow member 121, a flat member 122, and a space 123 open to bottom. The valve member 12 can be disposed in the chamber 113 through the bottom opening 112.

The valve member 12 can be oriented with bottom being open as that shown in FIGS. 6A and 6B prior to disposing in the chamber 113 through the opening 112. Thus, the flat member 122 separates the space 123 in the hollow member 121 from the chamber 113 of the floating member 11. Therefore, the chamber 113 of the floating member 11 and the valve member 12 having the flat member 122 disposed on top form a sealed hollow structure.

The valve member 12 can be disposed with either the flat member 122 on top or the space 123 open to top prior to disposing in the chamber 113 through the opening 112. Therefore, the chamber 113 of the floating member 11 and the valve member 12 disposed therein form a sealed hollow structure.

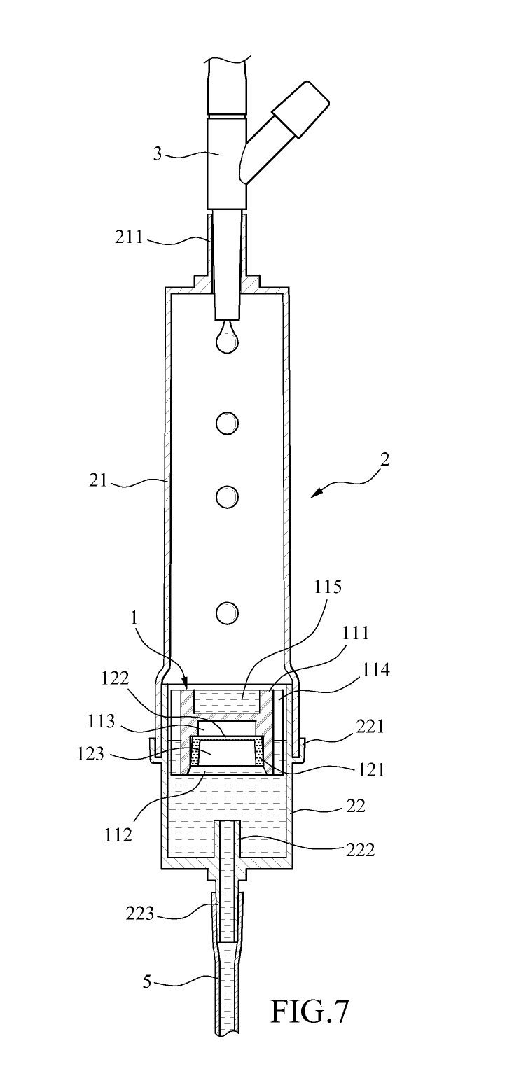

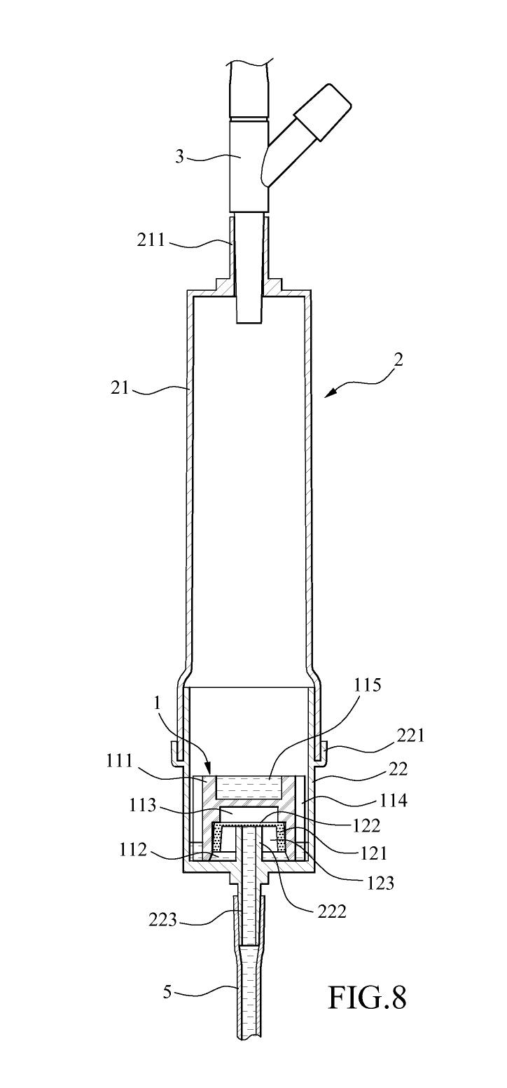

As shown in FIGS. 7 and 8 specifically, fluid can be fed from the drip chamber 4, the first plastic IV tubing 3, and the inlet 211 into the casing 2 to accumulate therein. As such, the floating member 11 and the valve member 12 as a unit float in the casing 2. In FIG. 7, it is shown that the recess 115 is filled with fluid and a portion of space of the casing 2 below the flow regulator 1 is filled with fluid. Level of the fluid in the casing 2 may drop due to fluid flow out of the casing 2 via the inner tubing connector 222, the outer tubing connector 223, and the second plastic IV tubing 5. In a position of FIG. 8, the inner tubing connector 222 is completely blocked by the flat member 122 of the valve member 12. As a result, the fluid flow is stopped. Alternatively, the valve member 12 can be disposed upside down in the chamber 113. Therefore, the chamber 113 of the floating member 11 and the valve member 12 disposed therein form a sealed hollow structure and together are adapted to float upward or downward in the casing 2.

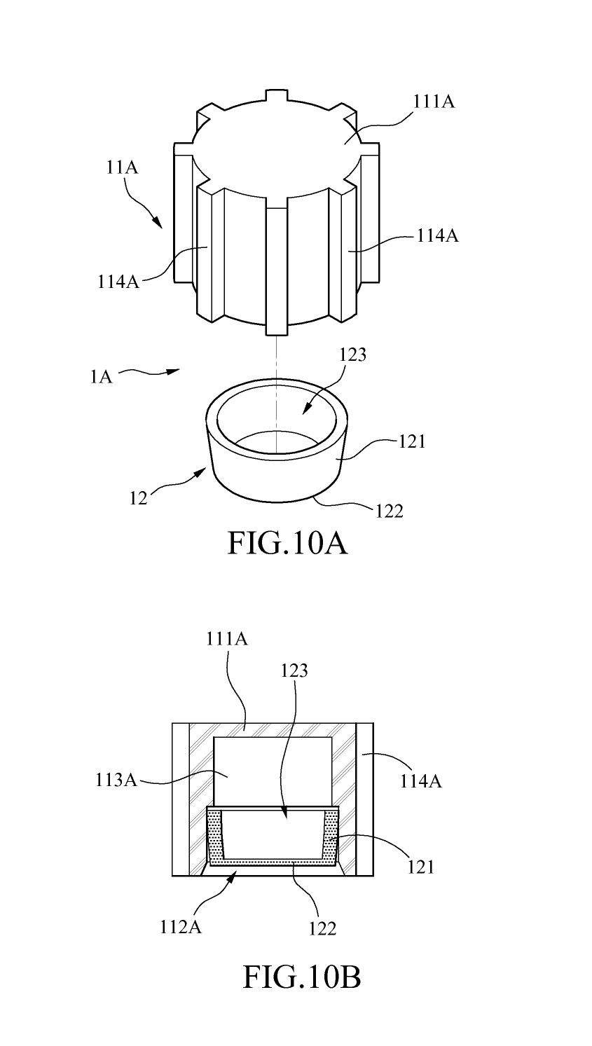

Referring to FIGS. 10A to 13, an intravenous flow control device in accordance with a second preferred embodiment of the invention is shown. The characteristics of the second preferred embodiment are substantially the same as that of the first preferred embodiment except the following:

A flow regulator 1A comprises a floating member 11A and a valve member 12A. The floating member 11A is shaped an inverted cup and comprises a hollow cylinder 111A, a smooth top, a bottom opening 112A on a bottom of the cylinder 111A, a stepped-diameter chamber 113A in the cylinder 111A and communicating with the bottom opening 112A, a plurality of equally spaced longitudinal ridges 114A of rectangular cross-section formed on an outer surface of the cylinder 111A.

As shown in FIGS. 10A and 10B specifically, in a first configuration the valve member 12 is bowl-shaped and comprises a downward tapered hollow member 121, a flat member 122, and a space 123 open to top. The valve member 12 can be disposed in a lower portion of the chamber 113A through the bottom opening 112A.

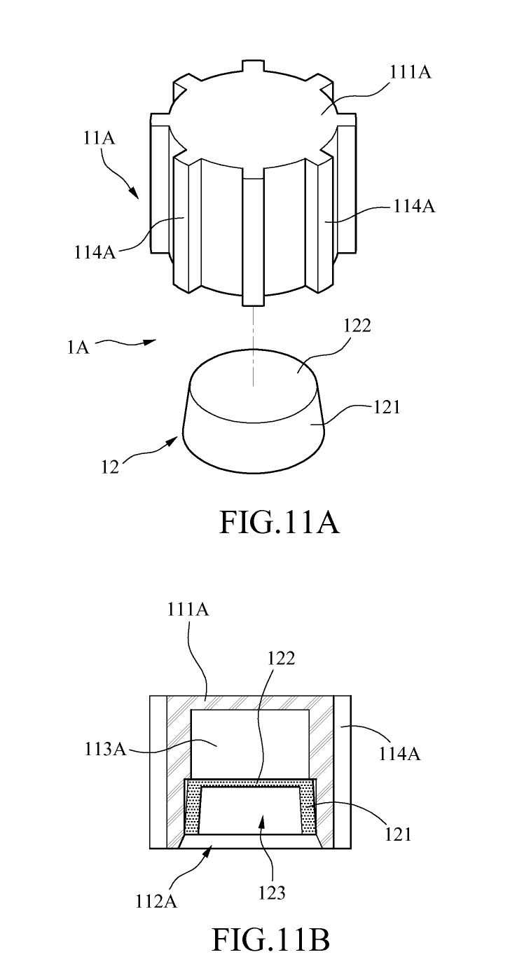

As shown in FIGS. 11A and 11B specifically, in a second configuration the valve member 12 is an inverted bowl-shaped member and comprises an upward tapered hollow member 121, a flat member 122, and a space 123 open to bottom. The valve member 12 can be disposed in a lower portion of the chamber 113A through the bottom opening 112A.

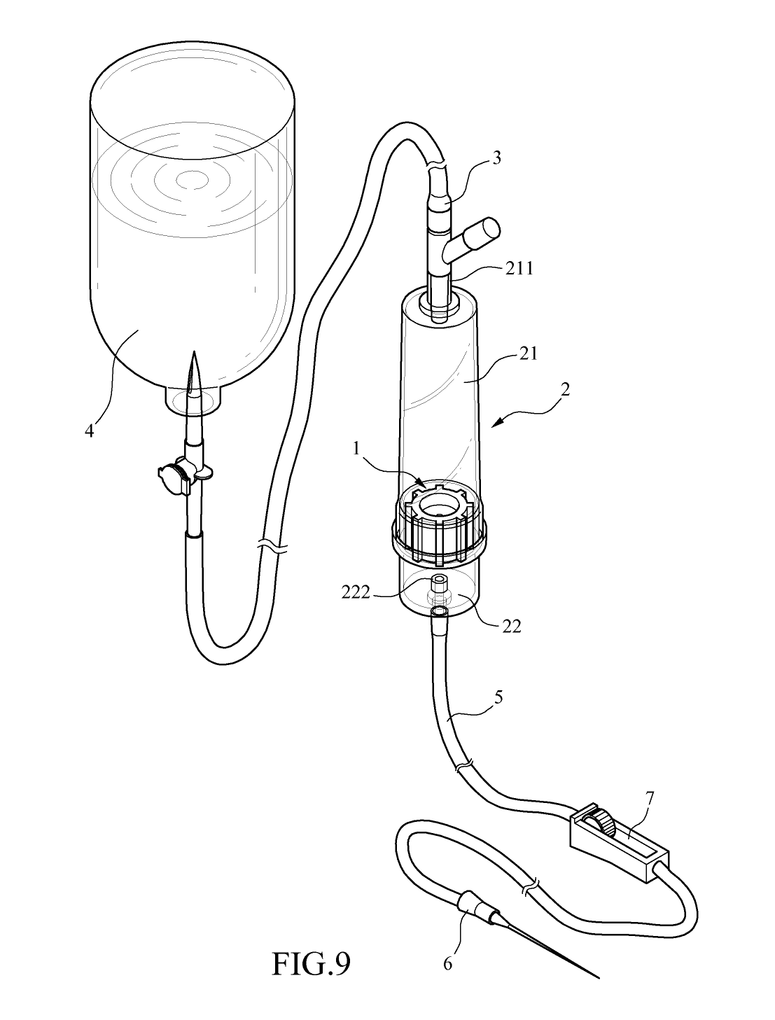

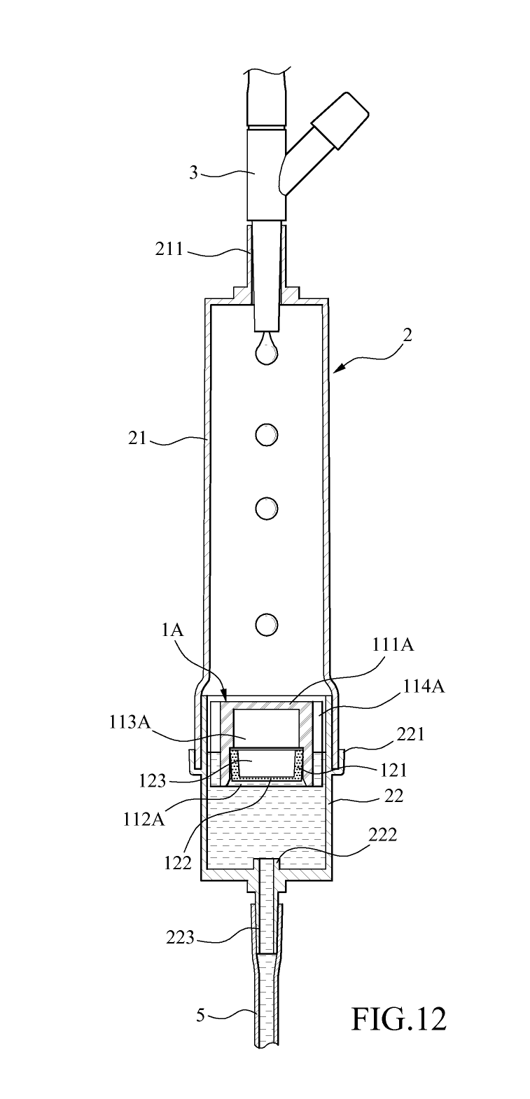

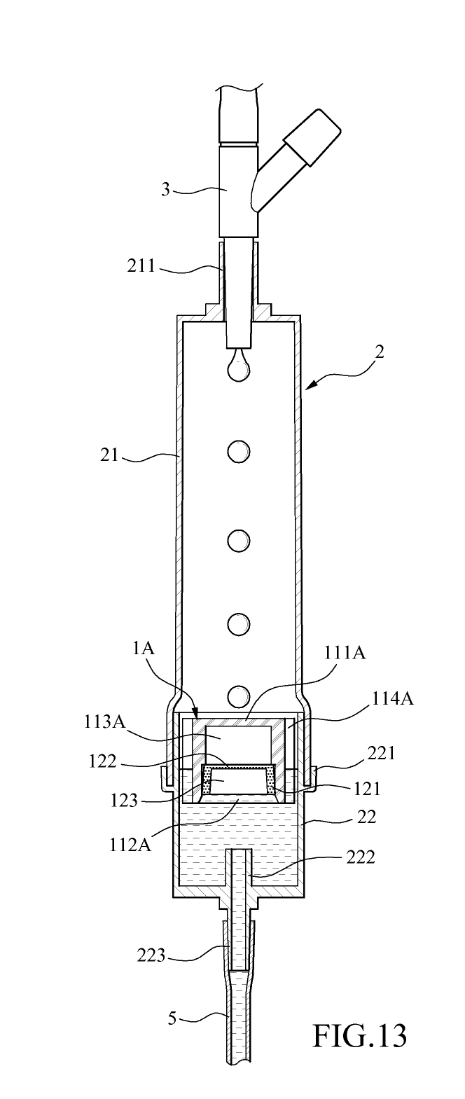

As shown in FIGS. 12 and 13 specifically in conjunction with FIG. 9, fluid can be fed from the drip chamber 4, the first plastic IV tubing 3, and the inlet 211 into the casing 2 to accumulate therein. As such, the floating member 11A and the valve member 12 as a unit float in the casing 2. In FIG. 12, it is shown that a portion of space of the casing 2 below the flow regulator 1 is filled with fluid. Level of the fluid in the casing 2 may drop due to fluid flow out of the casing 2 via the inner tubing connector 222, the outer tubing connector 223, and the second plastic IV tubing 5. In a position of FIG. 13, the inner tubing connector 222 is completely blocked by the flat member 122 of the valve member 12. As a result, the fluid flow is stopped.

The valve member 12 can be oriented with opening open to top as that shown in FIG. 12 or open to bottom as that shown in FIG. 13 as desired.

The valve member 12 can be oriented with the space 123 open to top as that shown in FIGS. 10A and 10B prior to disposing in the chamber 113 through the opening 112. Alternatively, the valve member 12 can be oriented with the flat member 122 on top as that shown in FIGS. 11A and 11B prior to disposing in the chamber 113 through the opening 112. Therefore, the chamber 113 of the floating member 11 and the valve member 12 disposed therein form a sealed hollow structure and together are adapted to float upward or downward in the casing 2.

Referring to FIGS. 12 and 13 again, the flow regulator 1A can float upward or downward in the casing 2 due to the sealed hollow structure formed by the chamber 113A of the floating member 11A and the valve member 12 therein.

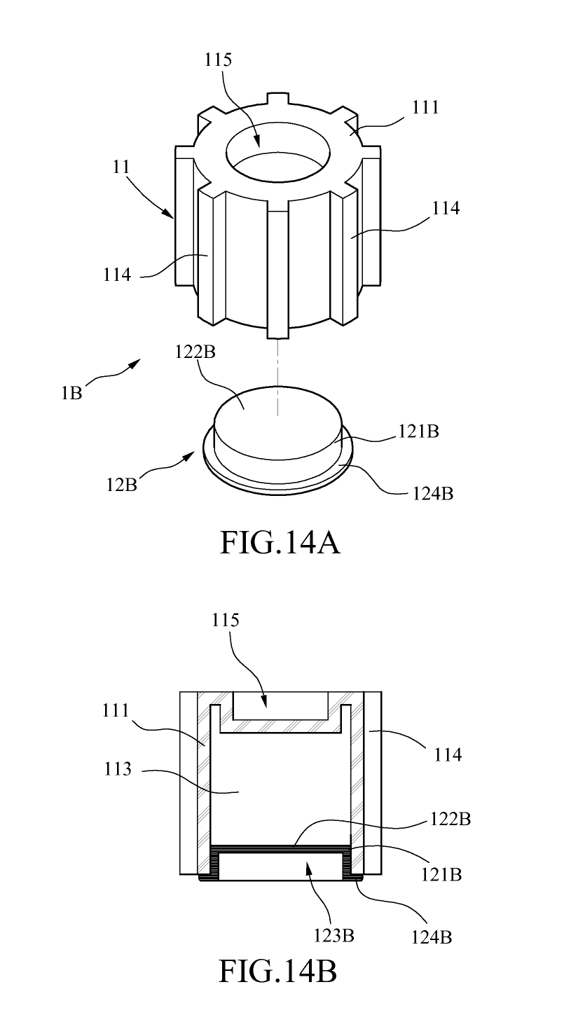

Referring to FIGS. 14A to 16, an intravenous flow control device in accordance with a third preferred embodiment of the invention is shown. The characteristics of the third preferred embodiment are substantially the same as that of the first preferred embodiment except the following:

A flow regulator 1B comprises a floating member 11 and a valve member 12B. The floating member 11 is a hollow cylindrical member and comprises a hollow cylinder 111, a bottom opening 112 on a bottom of the cylinder 111, a chamber 113 in a substantial portion of the cylinder 111 and communicating with the bottom opening 112, a plurality of equally spaced longitudinal ridges 114 of rectangular cross-section formed on an outer surface of the cylinder 111, and a recess 115 on a top of the cylinder 111. The recess 115 is separated from the chamber 113 by a member.

As shown in FIGS. 14A and 14B specifically, the valve member 12B is inverted cup shaped and comprises a hollow member 121B, a top 122B, an externally extended rim 124B on a bottom, and a space 123B open to bottom. The valve member 12 can be disposed in a lower portion of the chamber 113.

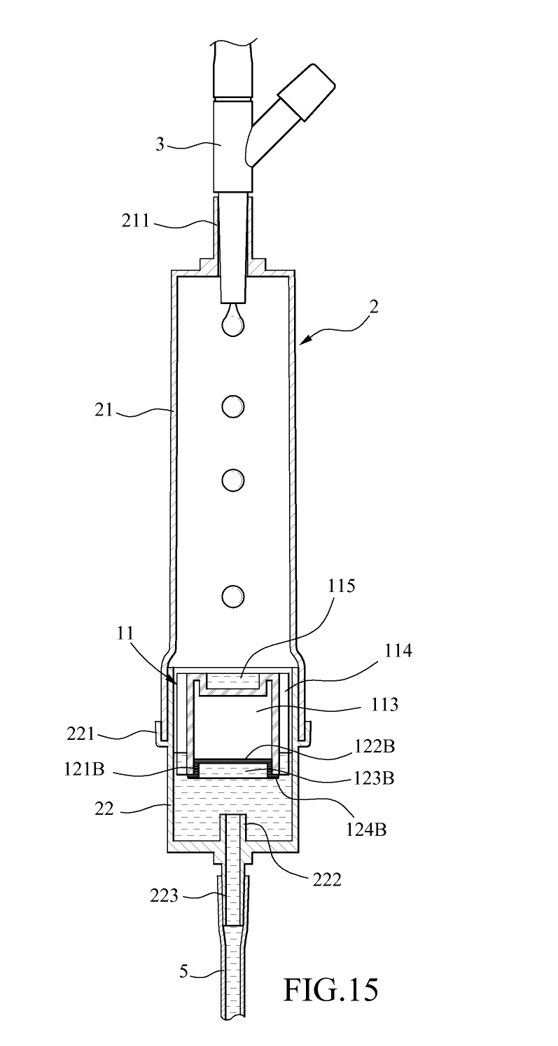

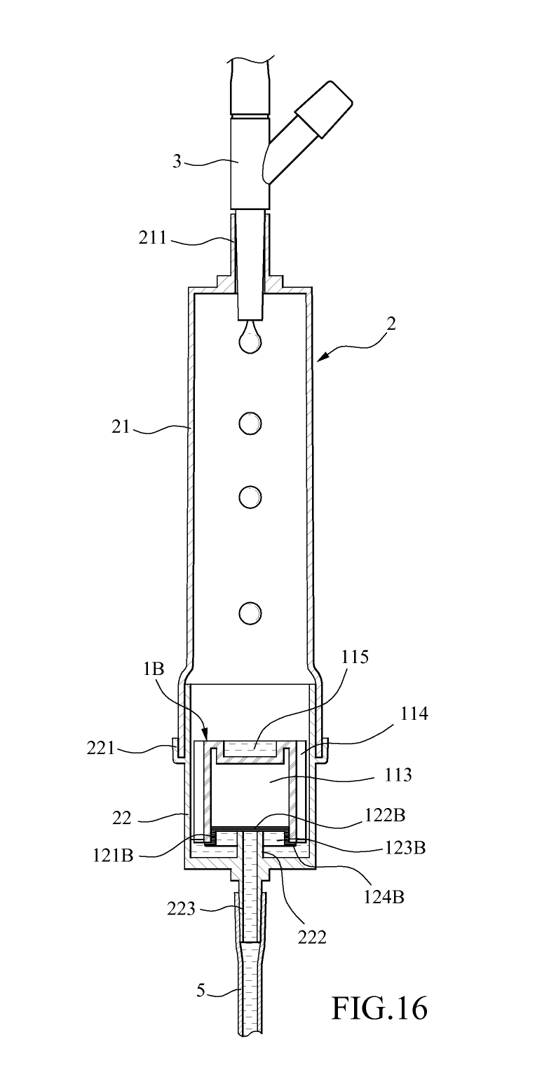

As shown in FIGS. 15 and 16 specifically in conjunction with FIG. 9, fluid can be fed from the drip chamber 4, the first plastic IV tubing 3, and the inlet 211 into the casing 2 to accumulate therein. As such, the floating member 11 and the valve member 12B as a unit float in the casing 2. In FIG. 15, it is shown that the recess 115 is filled with fluid and a portion of space of the casing 2 below the flow regulator 1 is filled with fluid. Level of the fluid in the casing 2 may drop due to fluid flow out of the casing 2 via the inner tubing connector 222, the outer tubing connector 223, and the second plastic IV tubing 5. In a position of FIG. 16, the inner tubing connector 222 is completely blocked by the bottom 122B of the valve member 12B. As a result, the fluid flow is stopped.

While the invention has been described in terms of preferred embodiments, those skilled in the art will recognize that the invention can be practiced with modifications within the spirit and scope of the appended claims.

What is claimed is:

1. An intravenous flow control device comprising:
a flow regulator (1) disposed in a casing (2) and comprising a floating member (11) including a hollow cylinder (111), an opening (112) on a bottom of the hollow cylinder (111), and a lower stepped-diameter chamber (113) in the hollow cylinder (111) and communicating with the opening (112); and a valve member (12) including a hollow member (121), a flat member (122), and a space (123) open to top;
wherein the valve member (12) is retained in the chamber (113) through the opening (112) and together they form a sealed hollow structure; and
wherein the flow regulator (1) floats on fluid in the casing (2) due to the sealed hollow structure.

2. The intravenous flow control device of claim 1, wherein the valve member (12) is shaped as an inverted bowl having a flat member (122) on a top so that the valve member (12) is retained in the chamber (113) through the opening (112) and together they form a sealed hollow structure.

3. The intravenous flow control device of claim 1, wherein the valve member (12) is shaped as a bowl having the space (123) open to top so that the valve member (12) is retained in the chamber (113) through the opening (112) and together they form a sealed hollow structure.

4. The intravenous flow control device of claim 1, wherein the casing (2) comprises an upper member (21), a lower member (22) fastened together, a fluid entrance (211), and a fluid exit (222).

5. The intravenous flow control device of claim 1, wherein the floating member (11) further comprises a recess (115) on a top of the hollow cylinder (111).

6. The intravenous flow control device of claim 5, wherein depth of the recess (115) is about one-fifth of height of the hollow cylinder (111).

7. The intravenous flow control device of claim 1, wherein the floating member (11) further comprises a plurality of equally spaced longitudinal ridges (114) formed on an outer surface of the hollow cylinder (111).

8. The intravenous flow control device of claim 1, wherein the valve member (12B) comprises an externally extended rim (124B) on a bottom, and a space (123B) open to the bottom, and wherein a lower portion of the hollow cylinder (111) is disposed on the externally extended rim (124B) by engaging the valve member (12B).
